(19) United States
(12) Patent Application Publication
(10) Pub. No.: US 2009/0022794 A1
(43) Pub. Date: Jan. 22, 2009

(54) Topiramate Tablet Formulation

(76) Inventor: Fjalar Johannson, Reykjavik (IS)

Correspondence Address: WENDEROTH, LIND & PONACK, L.L.P., 2033 K STREET N. W., SUITE 800, WASHINGTON, DC, 20006-1021, US

(21) Appl. No.: 11/886,548

(22) PCT Filed: Mar. 17, 2006
(86) PCT No.: PCT/IS2006/000006

§ 371 Date: Oct. 22, 2007

(30) Foreign Application Priority Data
Mar. 17, 2005 (IS) 7748

(51) Int. Cl. A61K 9/20; A61K 31/357

(52) U.S. Cl. 424/465; 424/464; 514/452

(57) ABSTRACT

The invention provides pharmaceutical compositions comprising as active ingredient topiramate, which are suitable for manufacturing tablet formulations by direct compression. The compositions preferably comprise spray-dried granulated mannitol and provide tablets of desired friability and hardness.

### FIELD OF INVENTION

The present invention describes a formulation for solid tablet dosage forms containing topiramate and a process for producing said formulation. The dosage form is obtained by direct compression.

### BACKGROUND

The active drug topiramate is an anticonvulsant, intended for use as an antiepileptic drug. The exact mode of action of the drug is not known but it is shown to effectively calm neuronal activity and reduce epileptic seizures. It is considered a broad spectrum anti-epileptic drug (AED) because it works to prevent both partial onset and generalized seizures. The drug may also be useful for treating conditions including seizures, mood disorders, post traumatic stress syndrome (PTSD), bipolar disorder, mania (all forms, such as acute mania, severe treatment-refractory mania, bipolar mania, etc.), depression, personality disorders, bipolar mood instability, schizophrenia, psychosis, bipolar spectrum disorders, rapid-cycling bipolar disorders, etc. Topiramate is also useful for treating patients with mood disorders that have not been adequately controlled by other medications, such as lamotrigine and gabapentin, and for treating patients with bipolar mood disorders that have not responded to lithium and/or other mood-stabilizers.

Topiramate is the nonproprietary name for the compound 2,3:4,5-Bis-O-(1-methylethylidene)-β-D-fructopyranose sulfamate, having the molecular formula C₁₂H₂₁NO₈S and the structural formula shown as Formula (I).

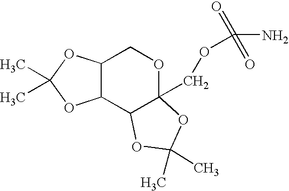

Topiramate is a white crystalline powder soluble in alkaline solutions as well as in acetone, dimethylsulfoxide and ethanol, while the solubility in water is 9.8 mg/mL. Tablets containing topiramate are marketed under the trademark Topamax® by Ortho-McNeil Pharmaceuticals. Topiramate may be produced according to the processes disclosed in U.S. Pat. Nos. 4,513,006 and 5,387,700. The compound has a very bitter taste and is sensitive to humidity, which causes degradation of the active compound. Degradation of topiramate can be readily detected by changes in appearance (discoloration) and the formation of sulfate ions which can be readily detected, e.g. by HPLC.

To enhance the stability of topiramate tablets, the prior art has suggested the use of blister packaging (see, EP1284711). Commercially available topiramate tablets are frequently packaged in the particular blister packages described in EP1284711, which require careful drying of the tablets prior to packaging. WO 2004/054547 suggests making bi- or multiphasic tablets comprising in at least one of the phases a hygroscopic gum material, e.g. xanthan gum, and containing topiramate as the active ingredient in another phase than the gum material.

As is well known in the art the three general processes for making compressed tablets are wet granulation, direct compression, and dry granulation (slugging or roller compaction). The method of preparation and type of excipients are selected to allow rapid compression of the tablets, and to provide tablets with desired and necessary attributes with respect to appearance, hardness, disintegrating ability, and an acceptable dissolution profile. Choice of fillers and other excipients will depend on the chemical and physical properties of the active ingredient, required behavior of the mixture during processing, and the desired properties of the final tablets.

The dry granulation method may be used where one of the constituents, either the active ingredient or the diluent, has sufficient cohesive properties to be tableted. The method consists of blending, slugging the ingredients, dry screening, lubrication, and compression.

The wet granulation method is used to convert a powder mixture into granules with suitable flow and cohesive properties for tableting. The procedure consists of mixing the powders in a suitable blender followed by adding the granulating liquid under shear to the mixed powders to obtain a granulation. The damp mass is then screened through a suitable screen and dried by tray drying or fluidized bed drying. Alternatively, the wet mass may be dried and passed through a mill. The overall process includes: weighing, dry powder blending, wet granulating, drying, mining, blending lubrication and compression. In general, powders do not have sufficient adhesive or cohesive properties to form hard, strong granules. Therefore, a binder is usually required to bond the powder particles together. However, heat sensitive drugs can usually not be compounded using wet granulation. Unfortunately, when compounding tablets using wet granulation, the large number of processing steps and processing time inherently increases the overall manufacturing costs.

Compounding tablets using direct compression, is a relatively quick process where the powdered materials are compressed directly without changing the physical and chemical properties of the drug. The active ingredient(s), excipients which can include auxiliary substances, such as a glidant and lubricant, are blended in a suitable blender before being compressed into tablets. This type of mixing was believed to be essential in order to prepare “pharmaceutically acceptable” dosage forms. For example, Remington's Pharmaceutical Sciences (RPS), pp 1203-1932 17th Ed. (1985), cautions pharmaceutical scientists that the manner in which a lubricant is added to a formulation must be carefully controlled. Accordingly, lubricants are usually added to a granulation by gentle mixing. RPS warns that prolonged blending of a lubricant with a granulation mix can materially affect hardness and disintegration time for the resulting tablets. Furthermore, Ansel et al. (1995) Pharmaceutical Dosage Forms and Drug Delivery Systems, 6th Ed. p. 199, indicate that excessive blending of lubricants with the granulate ingredients cause water proofing of the granule and reduces tablet hardness or strength of the compressed tablet.

The advantages of direct compression, are that few manufacturing steps are involved, (i.e. the overall process involves weighing of powders, blending and compression), and the elimination of heat and moisture.

Pharmaceutical manufacturers generally prefer direct compression techniques over wet or dry granulation methods because of quick processing time and low manufacturing cost. However, direct compression is usually limited to those situations where the drug or active ingredient has a crystalline structure and physical characteristics required to form pharmaceutically acceptable tablets. One or more excipients are combined with the active ingredient before the direct compression method can be used, since few if any active ingredients harbor all the collective properties required for a finished pharmaceutical dosage form. Unfortunately, with each added excipient to the formulation, the tablet size of the final product increases, and consequently manufacturers are often limited to using the direct compression method in formulations designed for low dosage forms.

A high strength dosage form, can only be compounded if the active ingredient harbors physical characteristics (e.g. cohesiveness), which negates the need of harboring some other excipients. Consequently, tablet homogeneity can be a problem for certain ingredients owing to segregation, and there is little possibility for prior wetting of a hydrophobic compound and subsequent dissolution enhancement, which is an effect obtained in wet granulation. For these reasons, direct compression is typically not a suitable formulation method for hydrophobic compounds that make up a substantial component (more than 10% wt, such as in particular 25 wt % or more) of the total formulation.

Stable economical topiramate tablets formulations obtainable by direct compression having a long shelf life and not requiring extensive pre-drying or specialized packaging would be much appreciated in the field.

### SUMMARY OF INVENTION

The present inventor has surprisingly found that a topiramate formulation as disclosed herein, can be compounded into tablets by direct compression, with good friability and homogeneity. The tablet formulation of the invention comprises in the range of about 5-35 wt % topiramate and in useful embodiments a large relative amount of topiramate, such as in the range of 15-35 wt %, e.g. about 15 wt % or 20 wt %. The formulation further comprises in the range of about 25-70 wt % spray-dried granulated mannitol and preferably a further excipient such as a diluent, a disintegrant and/or a lubricant.

### DETAILED DESCRIPTION

Terms

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the following specification and attached claims are approximations that may vary depending upon the desired properties sought to be obtained by the present invention. Generally, if not indicated otherwise, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. The following examples are intended to illustrate the invention without limiting the scope as a result.

As used in this invention, “topiramate” means the sulfamate-substituted monosaccharide shown above as Formula I. However, in the context herein pharmaceutically acceptable derivatives of topiramate are also encompassed by the term “topiramate”.

The topiramate can be in a crystalline phase, an amorphous phase, a semi-crystalline phase, a semi-amorphous phase, or comprise any mixture thereof.

The tablets of the invention preferably have a friability which is less than 1%, when tested according to the outlines provided by the European Pharmacopoeia. The hardness of the tablets can range from 30 to 200 N for the dosage forms.

Pharmaceutical Excipients

Pharmaceutical compositions according to the invention may comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents/diluents are microcrystalline cellulose such as Avicel® PH101, Avicel® PH102 and Emocel®90; lactose such as lactose monohydrate, lactose anhydrous, spray dried lactose, xylitol, Pharmatose® DCL21; dibasic calcium phosphate such as Emcompress®; mannitol; various starches; sorbitol; inositol; dextrose; sucrose; saccharides including glucose and/or mixtures of any of the foregoing. In some embodiments the formulation of the invention comprises in the range of about 10-35 wt % microcrystalline cellulose, such as in the range of about 10-25 wt %, or in the range of about 10-20 wt %, such as about 10 wt % or about 20 wt % microcrystalline cellulose, preferably of a type such a mentioned above.

In the composition of the invention granulated spray-dried mannitol is used as diluent. Mannitol is non-hygroscopic as it picks up less than 1% moisture at relative humidity as high as 90%. The disadvantage of mannitol is that it has poor flow properties and requires usually higher lubricant and glidant values. Granulated mannitol however usually gives a better flow than normal mannitol. Granulated spray-dried mannitol sold as Peariltol® SD 200 is particularly suitable for the direct compression formulation of the invention. The formulation of the invention comprises in the range of about 25-70 wt % granulated spray-dried mannitol, preferably in the range of about 35-65 wt %, such as in the range of 40-65 wt %, and more preferably in the range of about 44-65 wt %, such as in the range of about 44-55 wt %, e.g. about 44 wt % or about 50 wt % of granulated spray-dried mannitol.

Pregelatinised starch (e.g. Starch 1500) is preferred as a diluent, used in combination with the granulated spray-dried mannitol. It is a free-flowing and a directly compressible cornstarch. Starch 1500 is self-lubricating and self-disintegrating when compressed alone, but when combined with as little as 5-10% of an ingredient that is not self-lubricating it requires an additional lubricant. It contains about 10% moisture and is susceptible to softening when combined with excessive amounts (greater than 0.5%) of magnesium stearate. If included in the formulations of the invention, pregelatinised starch may comprise in the range of 4-15 wt %, such as in the range of about 6-10 wt %, e.g. about 8, 9 or 10 wt % of the formulation.

Other useful binders for the compositions of the invention are for example sucrose, glucose, cellulose derivatives, polyvinyl pyrrolidone (PVP), hydroxymethylcellulose, ethylcellulose, tragacanth, gelatin, sodium alginate, polymetacrylates, pregelatinized starch and hydroxypropylcellulose.

The tablet formulations may also comprise a disintegrant which accelerates the release of the active compound, such as for example one or more of a substance from the group of starches, including modified starches, e.g. crosslinked, such as sodium-starch glycolates, croscarmellose sodium, polyvinyl pyrrolidones, including modified polyvinyl pyrroildones, e.g. crosslinked, such as polyplasdone, crospovidone; celluloses, such as sodium and calcium carboxymethyl celluloses, modified celluloses, e.g. crosslinked; such as AcDiSol or any mixture of the above . . . of these are preferred crosslinked Na carboxymethyl cellulose, e.g. AcDiSol, polyvinyl pyrrolidone, e.g. cross-linked, e.g. polyplasdone and crospovidone. Such disintegrant preferably comprises in the range of about 2-10 wt % of the formulation of the invention, more preferably in the range of about 2-5 wt %, such as about 2.5 wt % or about 3 wt %.

Examples of useful lubricants are sodium stearate, waxes, calcium stearate, stearic acid, talc, magnesium stearate, hydrogenated vegetable oil, boric acid, sodium chlorate, carbowax 4000 and 6000, sodium oleate, sodium acetate, magnesium lauryl sulfate, sodium benzoate, DL-leucine, sodium benzoate and sodium lauryl sulfate.

The formulation also allows for the incorporation of glidants, for example but not limited to talc and cornstarch and Aerosil® (silica colloid anhydrous).

The tablet formulation of the invention may be wholly or partly covered by a coating layer, which may be a protective layer to prevent ingress of moisture or to prevent damage to the core of the tablet. By way of example, the following useful coating substances may be mentioned: methylcellulose, hydroxypropyl methylcellulose, PVP (Povidone), ethylcellulose (Ethocel 10 CPS), EUDRAGIT E 30D, EUDRAGIT L 30D, PHARMACOAT 606 6CPS, OPADRY, COTERIC, cellulose acetate phthalate. Preferred coating materials comprise hydroxypropylmethylcellulose and polyethylene glycol, with titanium dioxide as an opacifying agent. Other film-coating substances an methods well known to those of skill in the art may as well be employed.

Direct Compression Process

In another aspect, the invention provides a process for producing a topiramate tablet formulation as is described herein above, by direct compression. The process generally comprises mixing to homogeneity in suitable ratios as mentioned above topiramate, spray-dried granulated mannitol and optionally further excipients such as a diluent, a disintegrant and/or a lubricant, to obtain a composition such as described herein above; said composition may be sieved once or more to remove agglomerates; and compressing in a tableting machine with a tableting punch of suitable size tablets with a desired dose of topiramate. In some embodiments the active ingredient, diluent(s) and optionally a disintegrant are mixed to homogeneity, after which a lubricant is preferably admixed to the mix, and preferably the mixture is sieved again prior to compression to obtain tablets.

The following examples illustrate the present invention, however, it is not intended that the examples define or limit the scope of the invention, and it is to be understood that various other embodiments and modifications of the present invention are apparent to a skilled person in the art, without departing from the scope and the spirit of the invention as described.

### EXAMPLES

### Example 1

### Direct Compression Formulation for 25, 50, 100 and 200 mg Dose Tablets

| | | 25 mg | | 50 mg | | 100 mg | | 200 mg | |
|---|---|---|---|---|---|---|---|---|---|
| | Materials | tablet | | tablet | | tablet | | tablet | |
| 1 | Topiramate | 25 | mg | 50 | mg | 100 | mg | 200 | mg |
| 2 | Granulated spray-dried mannitol | 38.1 | mg | 76.2 | mg | 152.4 | mg | 304.8 | mg |
| 3 | Starch pregelatinised, Starch 1500 | 8.55 | mg | 17.1 | mg | 34.2 | mg | 68.4 | mg |
| 4 | Microcrystalline cellulose | 19 | mg | 38 | mg | 76 | mg | 152 | mg |
| 5 | Croscarmellose sodium | 2.85 | mg | 5.7 | mg | 11.4 | mg | 22.8 | mg |
| 6 | Silica colloidal anhydrous | 0.36 | mg | 0.72 | mg | 1.44 | mg | 2.88 | mg |
| 7 | Magnesium stearate non bovine | 1.14 | mg | 2.28 | mg | 4.56 | mg | 9.12 | mg |
| | Total weight | 95 | mg | 190 | mg | 380 | mg | 760 | mg |

Manufacturing Process

Ingredients 1-6 were mixed and sieved. Ingredient 7 was sieved and mixed with the blend. Samples were taken to assess homogeneity.

The powder was compressed with suitable punches for the different tablets sizes (hardness was adjusted in order to obtain friability of <1% after 400 rev.).

The uniformity of content for topiramate in the tablets was acceptable, less than 2.0% RSD, typically within the range of 1.0-1.5%.

For 25 mg tablets, the hardness was in the range of 30-60N, average about 45N; for 50 mg tablets hardness was in the range of 40-90N, average about 80N; for 100 mg tablets the hardness was in the range of 50-140N, average about 100N; for 200 mg tablets the hardness was in the range of 100-200N, average about 160N.

Tablets were spray-coated with a target weight increase of 4.0%, with Opadry® II (Colorcon, West Point, Pa., USA), different colors for the different doses.

In Examples 2-10, the ingredients were mixed substantially as described in Example 1 and tablets are compressed by direct compression, and optionally coated as described above in Example 1. All compositions shown below can be compounded to any of the above tablet sizes.

### Example 2

### Alternative Composition, Shown for 25 mg Tablets

| | Materials | 25 mg tablet | |
|---|---|---|---|
| 1 | Topiramate | 25 | mg |
| 2 | Granulated spray-dried mannitol | 64 | mg |
| 3 | Microcrystalllne cellulose | 10 | mg |
| 4 | Magnesium stearate non bovine | 1 | mg |
| | Total weight | 100 | mg |

### Example 3

### Alternative Composition, Shown for 25 mg Tablets

| | Materials | 25 mg tablet | |
|---|---|---|---|
| 1 | Topiramate | 25 | mg |
| 2 | Granulated spray-dried mannitol | 51 | mg |
| 3 | Microcrystalline cellulose | 20 | mg |
| 4 | Croscarmellose sodium | 3 | mg |
| 5 | Magnesium stearate non bovine | 1 | mg |
| | Total weight | 100 | mg |

### Example 4

### Alternative Composition, Shown for 100 mg Tablets

| | Materials | 100 mg tablet | |
|---|---|---|---|
| 1 | Topiramate | 100 | mg |
| 2 | Granulated spray-dried mannitol | 202.8 | mg |
| 3 | Microcrystalline cellulose | 80 | mg |
| 4 | Croscarmellose sodium | 12 | mg |
| 5 | Silica colloid anhydrous | 1.2 | mg |
| 6 | Magnesium stearate non bovine | 4 | mg |
| | Total weight | 400 | mg |

### Example 5

### Alternative Composition, Shown for 25 mg Tablets

| | Materials | 25 mg tablet | |
|---|---|---|---|
| 1 | Topiramate | 25 | mg |
| 2 | Granulated spray-dried mannitol | 51 | mg |
| 3 | Microcrystalline cellulose, | 20 | mg |
| 4 | Sodium starch glycolate | 3 | mg |
| 5 | Silica colloid anhydrous | 0.3 | mg |
| 6 | Magnesium stearate non bovine | 1 | mg |
| | Total weight | 100.3 | mg |

### Example 6

### Alternative Composition, Shown for 25 mg Tablets

| | Materials | 25 mg tablet | |
|---|---|---|---|
| 1 | Topiramate | 25 | mg |
| 2 | Granulated spray-dried mannitol | 48 | mg |
| 3 | Microcrystalline cellulose | 20 | mg |
| 4 | Povidone | 3 | mg |
| 5 | Croscarmellose sodium | 3 | mg |
| 6 | Magnesium stearate non bovine | 1 | mg |
| | Total weight | 100 | mg |

### Example 7

### Alternative Composition, Shown for 25 mg Tablets

| | Materials | 25 mg tablet | |
|---|---|---|---|
| 1 | Topiramate | 25 | mg |
| 2 | Granulated spray-dried mannitol | 44.7 | mg |
| 3 | Microcrystalline cellulose | 25 | mg |
| 4 | Croscarmellose sodium | 3 | mg |
| 5 | Silica colloidal anhydrous | 0.3 | mg |
| 6 | Magnesium stearate non bovine | 2 | mg |
| | Total weight | 100 | mg |

### Example 8

### Alternative Composition, Shown for 25 mg Tablets

| | Materials | 25 mg tablet | |
|---|---|---|---|
| 1 | Topiramate | 25 | mg |
| 2 | Granulated spray-dried mannitol | 44.2 | mg |
| 3 | Microcrystalline cellulose | 25 | mg |
| 4 | Croscarmellose sodium | 3 | mg |
| 5 | Sodium Lauryl Sulfate | 0.5 | mg |
| 6 | Silica colloidal anhydrous | 0.3 | mg |
| 7 | Magnesium stearate non bovine | 2 | mg |
| | Total weight | 100 | mg |

### Example 9

### Alternative Composition, Shown for 25 mg Tablets

| | Materials | 25 mg tablet | |
|---|---|---|---|
| 1 | Topiramate | 25 | mg |
| 2 | Granulated spray-dried mannitol | 49.3 | mg |
| 3 | Microcrystalline cellulose | 9 | mg |
| 4 | Starch pregelatinize, Starch 1500 | 13.5 | mg |
| 5 | Croscarmellose sodium | 1.8 | mg |
| 6 | Silica colloidal anhydrous | 0.3 | mg |
| 7 | Magnesium stearate non bovine | 1.1 | mg |
| | Total weight | 100 | mg |

### Example 10

### Alternative Composition, Shown for 25 mg Tablets

| | Materials | 25 mg tablet | |
|---|---|---|---|
| 1 | Topiramate | 25 | mg |
| 2 | Granulated spray-dried mannitol | 32.38 | mg |
| 3 | Starch pregelatinized, Starch 1500 | 9.50 | mg |
| 4 | Microcrystalline cellulose | 23.75 | mg |
| 5 | Croscarmellose sodium | 2.85 | mg |
| 6 | Silica colloidal anhydrous | 0.38 | mg |
| 7 | Magnesium stearate non bovine | 1.14 | mg |
| | Total weight | 95 | mg |

1. A tablet formulation, produced by direct compression comprising:
in the range of about 5-35 wt % topiramate, and
in the range of about 25-70 wt % spray-dried granulated mannitol.

2. The tablet formulation of claim 1 comprising in the range of about 15-30 wt % topiramate.

3. The tablet formulation of claim 2 comprising in the range of about 20-30 wt % topiramate.

4. The tablet formulation of claim 1, each tablet comprising a topiramate dose selected from the group consisting of: about 25 mg, about 50 mg; about 100 mg; and about 200 mg.

5. The tablet formulation of an of claim 1, further comprising a disintegrant.

6. The tablet formulation of claim 5 comprising a disintegrant selected from a group consisting of cross-linked polyvinyl pyrrolidone, corn starch, modified starch, cross-linked sodium carboxymethylcellulose, sodium starch glycolate, and mixtures thereof.

7. The tablet formulation of claim 6 comprising cross-linked sodium carboxymethylcellulose as a disintegrant.

8. The tablet formulation of claims 1, further comprising a lubricant.

9. The tablet formulation of claim 8 comprising a lubricant selected from a group consisting of magnesium stearate, sodium stearate, stearic acid, calcium stearate, magnesium oleate, oleic acid, potassium oleate, sodium stearyl fumarate, magnesium palmitate, glyceryl dibehenate, and colloidal silicon dioxide and mixtures thereof.

10. The tablet formulation of claim 1 further comprising a filler selected from a group consisting of microcrystalline cellulose and pregelatinized starch.

11. The tablet formulation of claim 10 comprising in the range of about 10-35 wt % microcrystalline cellulose as a filler.

12. The tablet formulation of claim 1 comprising:
in the range of about 20-30 wt % topiramate,
in the range of about 35-65 wt % spray-dried granulated mannitol,
in the range of about 10-25 wt % microcrystalline cellulose, and
in the range of about 1-2 wt % magnesium stearate.

13. The tablet formulation of claim 12, further comprising in the range of about 0.2-0.5 wt % anhydrous colloidal silica.

14. The tablet formulation of claim 13 comprising:
about 26 wt % topiramate,
about 40 wt % spray-dried granulated mannitol,
about 9 wt % pregelatinized starch,
about 20 wt % microcrystalline cellulose,
about 3 wt % crosscarmellose sodium,
about 0.4 wt % anhydrous colloidal silica, and
about 1.4 wt % magnesium stearate.

15. The tablet formulation of claim 12 comprising:
about 25 wt % topiramate,
about 64 wt % spray-dried granulated mannitol,
about 10 wt % microcrystalline cellulose, and
about 1 wt % magnesium stearate.

16. The tablet formulation of claim 12 comprising:
about 25 wt % topiramate,
about 51 wt % spray-dried granulated mannitol,
about 20 wt % microcrystalline cellulose,
about 3 wt % crosscarmellose sodium, and
about 1 wt % magnesium stearate.

17. The tablet formulation of claim 15, further comprising about 0.3 wt % anhydrous colloidal silica.

18. A process for producing a topiramate tablet formulation, comprising the steps of mixing to homogeneity topiramate, granulated spray-dried mannitol, a disintegrant and a lubricant, and optionally one or more further excipients, wherein the topiramate comprises in the range of 5-35 wt % of the final composition, and said spray-dried mannitol comprises about 25-70 wt % of the final composition, optionally sieving the mix once or more to remove unwanted agglomerates, compressing the mix in a tablet punching machine with a tablet punch of suitable size to obtain compressed tablets of a size which will give tablets having a desired dose of topiramate.

19. The process of claim 18, wherein the topiramate comprises in the range of 15-30wt % of the final composition.

20. The process of claim 18, wherein the topiramate comprises in the range of 20-30wt % of the final composition.

21. The process of claim 18, wherein topiramate, spray-dried mannitol, said disintegrant and optionally said one or more further excipients are mixed to homogeneity, after which said lubricant is admixed to the mix.

22. The process of claim 18, wherein said disintegrant is selected from the group consisting of cross-linked polyvinyl pyrrolidone, corn starch, modified starch, cross-linked sodium carboxymethylcellulose, sodium starch glycolate, and mixtures thereof

23. The process of claim 18, wherein said disintegrant comprises cross-linked sodium carboxymethylcellulose

24. The process of claim 18, further comprising admixing in the range of about 10-25 wt % microcrystalline cellulose to the mixture.

25. The tablet of claim 16 further comprising about 0.3 wt % anhydrous colloidal silica.
